(19) United States
(12) Patent Application Publication
(10) Pub. No.: US 2022/0178936 A1
(43) Pub. Date: Jun. 9, 2022

(54) A SENSITIVE LC-MS ASSAY TO MEASURE CURCUMINOIDS IN COMPLEX BIOLOGICAL SAMPLES

(71) Applicant: THE REGENTS OF THE UNIVERSITY OF CALIFORNIA, Oakland, CA (US)

(72) Inventors: Kym Francis FAULL, Los Angeles, CA (US); Alexander Jaeoh YOON, Glendale, CA (US); Philip HAMPTON, Newbury Park, CA (US)

(73) Assignees: THE REGENTS OF THE UNIVERSITY OF CALIFORNIA, Oakland, CA (US); CALIFORNIA STATE UNIVERSITY CHANNEL ISLAND, Camarillo, CA (US)

(21) Appl. No.: 17/601,189

(22) PCT Filed: Apr. 14, 2020
(86) PCT No.: PCT/US2020/028131

§ 371 Date: Oct. 4, 2021

Related U.S. Application Data
(60) Provisional application No. 62/833,738, filed on Apr. 14, 2019.

(51) Int. Cl. G01N 33/64; G01N 1/28; G01N 1/40; G01N 30/72; G01N 30/86; G01N 30/08

(52) U.S. Cl. CPC G01N 33/64; G01N 1/286; G01N 1/4055; G01N 30/7233; G01N 2030/045; G01N 30/08; G01N 2001/2866; G01N 2001/4061; G01N 30/8679

(57) ABSTRACT

A sensitive method for quantitating curcuminoids in biological and other samples is described wherein curcuminoids in the sample are derivatized to boron difluoride curcuminoid complexes then analyzed by liquid chromatography-mass spectroscopy.

### BACKGROUND

Turmeric, a spice derived from the rhizome of Curcuma longa L., has been used in China and India for centuries to treat a variety of human ailments and physical wounds. The presumed major active component of turmeric is a yellow pleiotropic polyphenol called curcumin. In many studies, curcumin is reported to possess a wide range of bioactivities, such as anti-inflammatory, anti-oxidant, anti-proliferative, and anti-angiogenic effects, only to name a few. Demethoxycurcumin (DMC) and bis-demethoxycurcumin (BDMC) are other closely related bioactive polyphenols found in turmeric, which are structurally similar to curcumin only with loss of one or both phenyl o-methoxy groups, respectively. Findings from a number of studies have demonstrated bioactivities of DMC and BDMC, and in limited cases, their potency is equivalent to or even greater than those of curcumin. Also, independent studies found that DMC and BDMC can synergistically enhance the effect of curcumin, although the actual mechanism is unknown. Together called curcuminoids, curcumin, DMC, and BDMC are found in various compositions in turmeric-based dietary supplements as well as commercially available purified curcumin mixtures frequently used for biomedical research.

With the growing interest in the therapeutic and disease-preventing potentials of curcumin and its related compounds, numerous clinical trials have already been approved or are currently on-going. However, there is also skepticism toward actual benefits of curcuminoids in vivo. One major reason is their low bioavailability. Although several formulations are on the market today, and have been developed to improve the absorption, curcuminoids are still subjected to rapid glucuronidation and sulfate conjugation for clearance after crossing the submucosal or intestinal barriers. Therefore, in order to assess the pharmacological efficacy of curcuminoids and their metabolites, accurate measurement of unconjugated curcumin concentrations in complex biological samples, such as plasma and tissue, is critical, but it has been challenged by the limited detection sensitivity of existing assays.

It is toward improved and more sensitive detection methods for curcuminoids in biological samples that the present invention is directed.

### SUMMARY

In one embodiment, the method for quantifying one or more curcuminoids present in a sample is provided. The method comprising the steps of derivatizing the curcuminoids in the sample to a boron difluoride curcuminoid complex, and quantifying the boron difluoride curcuminoid complexes by liquid chromatography (LC)-electrospray ionization (ESI)-tandem mass spectrometry (MS/MS), thereby quantifying the one or more curcuminoids and their metabolites present in the sample. In one embodiment, the curcuminoids refer to curcumin, demethoxycurcumin, bisdemethoxycurcumin, and other derivatives and metabolites of curcumin containing a diketone moiety. In one embodiment, the method is capable of identifying small levels of curcuminoids, each at a calculated level of quantitation (LoQ) and detection (LoD) of at least 0.05 nM and 0.01 nM, respectively.

In one embodiment, the derivatizing is achieved by reacting the one or more curcuminoids in the sample with boron trifluoride. In one embodiment, the boron trifluoride is in the form of boron trifluoride diethyl etherate.

In some embodiments, curcuminoids are, by way of non-limiting examples, curcumin (abbreviated C), bis-demethoxycurcumin (abbreviated BDMC), and demethoxycurcumin (abbreviated DMC). Metabolites of curcuminoids are, by way of non-limiting examples, tetrahydrocurcumin (abbreviated TC) and glucuronidated curcuminoids curcumin β-D-glucuronide (abbreviated CG), demethoxycurcumin glucuronide (abbreviated DMCG) and bisdemethoxycurcumin glucuronide (abbreviated BDMCG). Other curcuminoids and curcuminoid metabolites that retain the diketone moiety are also embraced herein.

In one embodiment, at least one internal standard is added to the sample, such as but not limited to ²H₆-curcumin and/or ²H₆-tetrahydrocurcumin.

In one embodiment, the sample is a bodily fluid or fraction thereof, a tumor sample or specimen, a tissue sample or specimen, a plant, a foodstuff or extract thereof, a cosmetic, a food supplement or flavoring, or a dietary supplement. In one embodiment, the bodily fluid or fraction thereof is plasma, serum, whole blood, red blood cells (RBC), urine, lymphatic fluid, cerebrospinal fluid, saliva, sweat or semen.

In one embodiment, the method comprises the step of derivatizing the one or more curcuminoids in the sample with boron trifluoride, subjecting the sample to liquid chromatography separation and mass spectrometry. Variations on sample preparation prior to derivatization, sample handling after derivatization, and LC-MS analysis and data analysis are full embraced herein.

In one embodiment, the method comprises the steps of:
(1) adding at least one internal standard to the sample or to an extract thereof;
(2) treating the sample or extract thereof with methanol containing 10 mM acetic acid, mixing, and collecting the supernatant;
(3) drying down the supernatant;
(4) adding glacial acetic acid and derivatizing the one or more curcuminoids therein with boron trifluoride; and
(5) subjecting the sample to liquid chromatography separation and tandem mass spectrometry;
wherein the signal detected from each of the one or more curcuminoid in the sample or extract thereof compared to a signal from a standard, relative to the signal of at least one internal standard, indicates the quantity of each curcuminoid in the sample or extract thereof.

In certain embodiments, the method is modified to accommodate different types of samples.

In one embodiment, the liquid chromatography comprises a gradient wherein solvent A is water/formic acid 100/0.01 (vol/vol) and solvent B is acetonitrile/isopropanol 50/50 (vol/vol), comprising:

| Time | % A | % B |
|---|---|---|
| 0 | 85 | 15 |
| 1.5 | 85 | 15 |
| 6.5 | 0 | 100 |
| 7.5 | 0 | 100 |
| 8 | 85 | 15 |
| 10 | 85 | 15 |

In one embodiment, the liquid chromatography utilizes a C18 reversed phase column.

These and other aspects of the invention will be appreciated from the following brief description of the figures and ensuing detailed description of the invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 depicts the structure of curcumin and the BF₂-curcumin product that forms after treatment with boron trifluoride etherate (BF₃.OEt₂) under acidic conditions.

FIG. 2 shows negative ion electrospray mass spectra of curcumin (left) and BF₂-curcumin (right) collected by direct injections into an ESI source connected to a linear ion trap mass spectrometer. The concentration of the solutions used to collect these spectra were not the same with a more dilute solution used for the BF₂ derivative, and this is reflected in higher background in the spectrum.

FIG. 3 shows an expanded view of the ¹H-NMR (400 MHz) spectra of curcumin (top) and BF₂-curcumin (bottom) showing the structural symmetry and proton splitting patterns of the two molecules. BF₂-curcumin proton assignments were made with reference to the previously described curcumin proton assignments (http://sdbs.db.aist.go.jp, using CAS 458-37-7). Samples were dissolved in ²H₆-dimethylsulfoxide (1 mg/mL) and integrations were made relative to the j and j* signals.

FIG. 4 shows the stability of BF₂-curcuminoid complex tested to validate the assay for overnight analysis. Eight QC-mid samples were prepared, stored tightly capped to minimize contact with atmospheric moisture in the autosampler at 20° C., and each sample was analyzed every hour for up to seven hours. The data were assembled as the area of the peaks in each chromatogram.

FIG. 5 shows peak areas for curcumin (x symbols, m/z 368 parent->m/z 217 fragment) and BF₂-curcumin (circles, m/z 415 parent->m/z 400 fragment) using previously optimized LC/MS conditions for each. The data were obtained from plasma samples spiked with curcumin and processed as described in the examples. The peak areas for the BF₂-C signals were on average 28-fold greater than the peak areas for the C signals.

FIG. 6 shows relative responses for curcumin and its related compounds after 1 pmol of each was stored either in PBS (right) or human plasma (left), and the samples were subjected up to 4 freeze (−80° C.) and thaw (RT) cycles prior to LC-MS analysis. The internal standard was not used in this experiment, and the data is presented as % change in absolute peak areas relative to the zero hour time point.

FIG. 7 shows select regions of the reconstructed ion chromatograms for the curcuminoids in: A, human plasma spiked with standards at the practical LoQ (0.5 nM for C, DMC, BDMC, equivalent to 15 fmol of each compound injected assuming 100% recovery; B, extract of Day 17 plasma from a volunteer who orally consumed four tablets of a commercially available over-the-counter curcuminoid formulation (Longvida®, 260 mg curcuminoids per 1000 mg tablet) daily for 17 consecutive days; C, Extract from 83.8 mg of wet tumor tissue from a mouse that had been administered a proprietary turmeric preparation in development (PHRAD 129, Aveta Biomics, Bedford, Mass.).

FIG. 8 shows select regions of the reconstructed ion chromatograms for the curcuminoid metabolites in (same as FIG. 7): A, human plasma spiked with standards at the practical LoQ (5 nM for TC and CG, equivalent to 150 fmol of each compound injected assuming 100% recovery; B, human plasma from a volunteer who orally consumed four tablets of a commercially available over-the-counter curcuminoid formulation (Longvida®, 260 mg curcuminoids per 1000 mg tablet) daily for 17 consecutive days; C, Extract from 83.8 mg of wet tumor tissue from a mouse that had been administered a proprietary turmeric preparation in development (PHRAD 129, Aveta Biomics, Bedford, Mass.).

FIG. 9 shows select regions of the reconstructed ion chromatograms for the curcuminoids in: A, drug-naïve human plasma spiked only with the internal standard ²H₆ curcumin; B, Mouse brain extract from 70.2 mg of wet tissue; C, Mouse liver extract from 63.1 mg of wet tissue. The samples from B and C were obtained from animals that had been administered a proprietary turmeric preparation in development (PHRAD 129, Aveta Biomics, Bedford, Mass.).

FIG. 10 shows select regions of the reconstructed ion chromatograms for the curcuminoid metabolites in (same as FIG. 9): A, drug-naïve human plasma spiked only with the internal standard ²H₆ curcumin; B, Mouse brain extract from 70.2 mg of wet tissue; C, Mouse liver extract from 63.1 mg of wet tissue. The samples from B and C were obtained from animals that had been administered a proprietary turmeric preparation in development (PHRAD 129, Aveta Biomics, Bedford, Mass.).

FIG. 11 shows temporal profiles of the concentration of curcuminoids [curcumin (C), demethoxycurcumin (DMC) and bis-demethoxycurcumin (BDMC; left panels] and curcumin metabolites [tetrahydrocurcumin (TC) and C, DMC and BDMC glucuronides (CG, DMCG, and BDMCG, respectively); right panels] on Day 1 (upper panels) and Day 17 (lower panels) in the plasma of the volunteer who orally consumed four tablets of over-the-counter curcuminoid formulation (Longvida®, 260 mg curcuminoids per 1000 mg tablet) daily for 17 consecutive days. Blood was collected at 0, 1, 2, 3, 4, and 5 hours on each of Days 1 and 17. Data points are the mean+/−standard error of the mean from triplicate aliquots analyzed for each time point.

### DETAILED DESCRIPTION

The present subject matter may be understood more readily by reference to the following detailed description that forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom a sample may be obtained from in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

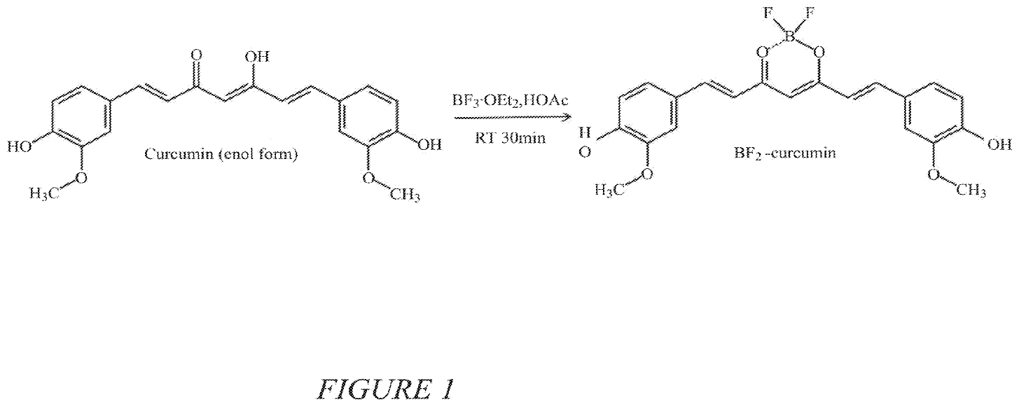

As noted above, sensitive quantitation of curcuminoids in samples is highly desirable. The inventors hereof addressed this issue by employing methods that convert curcuminoids such as but not limited to curcumin, demethoxycurcumin (DMC), and bisdemethoxycurcumin (BDMC), and their metabolites, into their respective boron difluoride (BF₂) complexes. The conversion reaction rapidly goes to completion at room temperature (FIG. 1), and the detection sensitivity of the BF₂-curcuminoid complex with electrospray ionization (ESI) mass spectrometry is significantly better than that of native molecules, with an, in one embodiment, on average of 28-fold increase in signal intensity. The reaction was optimized into a simple single-step process, and used it to develop a sensitive LC-MS assay to measure curcuminoids in, in one embodiment, plasma and soft tissues. The assay has been carefully validated, and may be applied to investigate the pharmacokinetics of curcumin (C), DMC, BDMC, tetrahydrocurcumin (TC) and curcumin-glucuronide (CG) in the plasma of individuals who are taking an over-the-counter curcumin formulation, and in murine brains taken from animals who are administered the same or similar formulations. Use of the assay for measuring curcuminoids and metabolites of curcuminoids in other bodily fluids and tissues, as well as in natural and in manufactured products is fully embraced herein. Encompassed herein are other methods of measuring boron difluoride curcuminoid complexes in the prepared samples, such as but not limited to GC-MS and LC-UV methods.

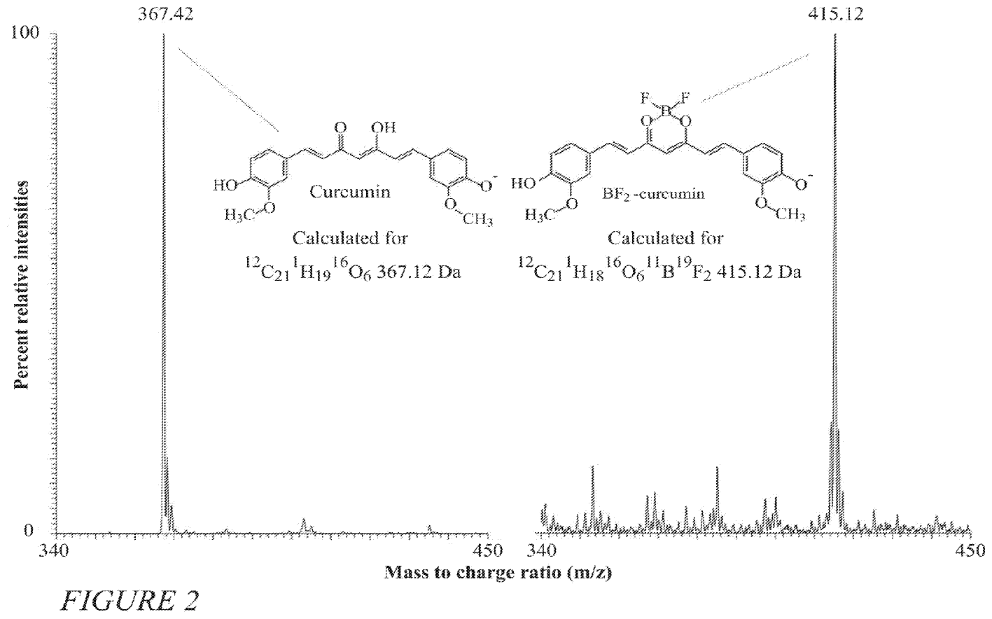

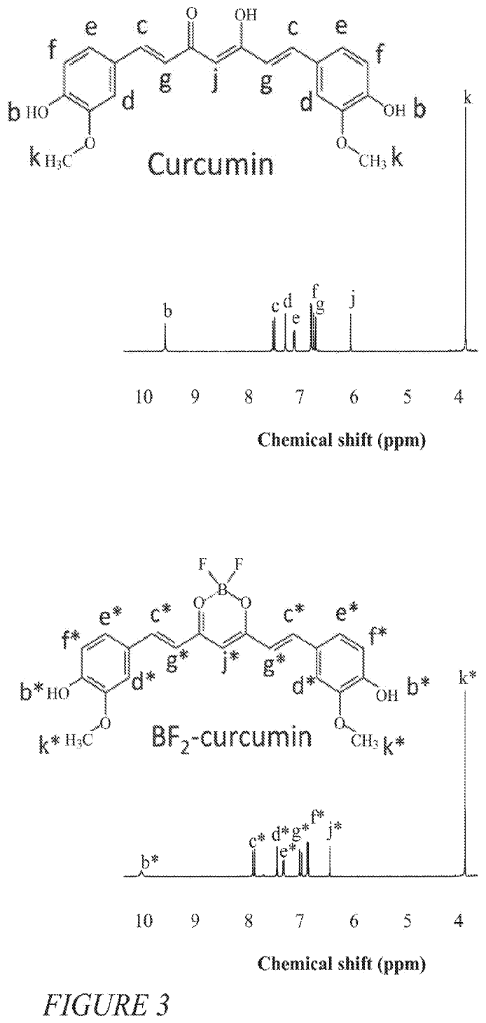

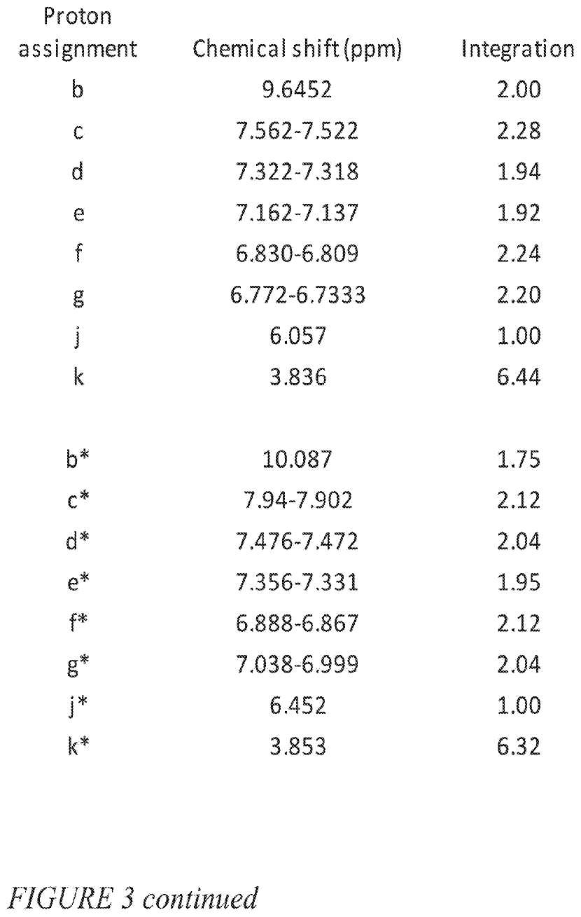

As will be seen in the examples below, the completion of the BF₂ complex formation was determined by comparing the mass and ¹H-NMR spectra of curcumin and the product after the reaction with BF₃ reagent. No residual signal for starting material was observed in either spectra following the reaction (FIGS. 2-3). The comparison of ¹H-NMR spectra of curcumin before and after the derivatization shows chemical shifts indicating the formation of BF₂-curcumin complex, which retains the structural symmetry and proton splitting patterns of the original molecule.

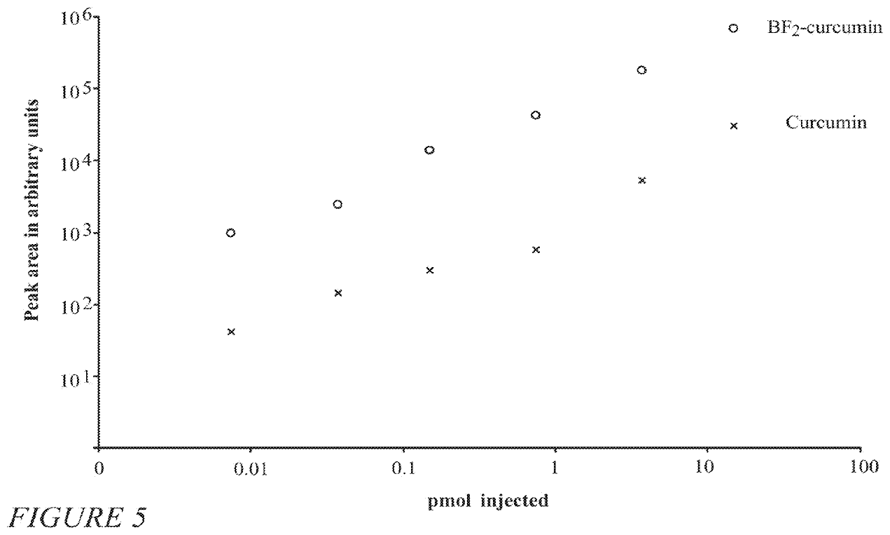

The ESI response for the BF₂-curcumin complex was on average 28-fold-greater than that from underivatized curcumin (FIG. 5). The binding of the BF₂ group to the diketone moiety appears to make the 4-hydroxy groups more acidic as shown in the H¹-NMR spectrum (FIG. 3, proton b and b*). Therefore, the BF₂-curcumin complex would ionize better under negative ESI-mode through loss of a proton from a 4-hydroxy group.

The assay as described here was shown to be robust and reproducible. From human plasma, the extraction recovery was within a range of 85%-118% for all compounds across all concentration levels (see Table 2). Accuracy and precision (see Table 3) shows a % Coefficient of Variation less than 15% and an accuracy between 88-109% for all compounds except for TC. TC was measured using d6C as internal standard. The lack of a heavy-isotope-labeled internal standard for this compound in this experiment may explain the slightly inferior TC results.

According to convention, the calculated limit of detection (LoD) and limit of quantitation (LoQ) of the assay were estimated from the mean background signal of blank control plus 3 or 10 times the standard deviation of the mean, respectively. Using these formulae, the calculated LoD and LoQ of C, DMC, and BDMC were 0.01 nM and 0.05 nM, respectively. For TC and CG, their calculated LoD and LoQ were 0.1 nM and 0.5 nM, respectively. However, to ensure maximal reliability and accuracy of quantitation, we used ten times the calculated LoQ as the practical LoQ (0.5 nM for C, DMC, and BDMC; and 5 nM for CG and TC) in the analysis.

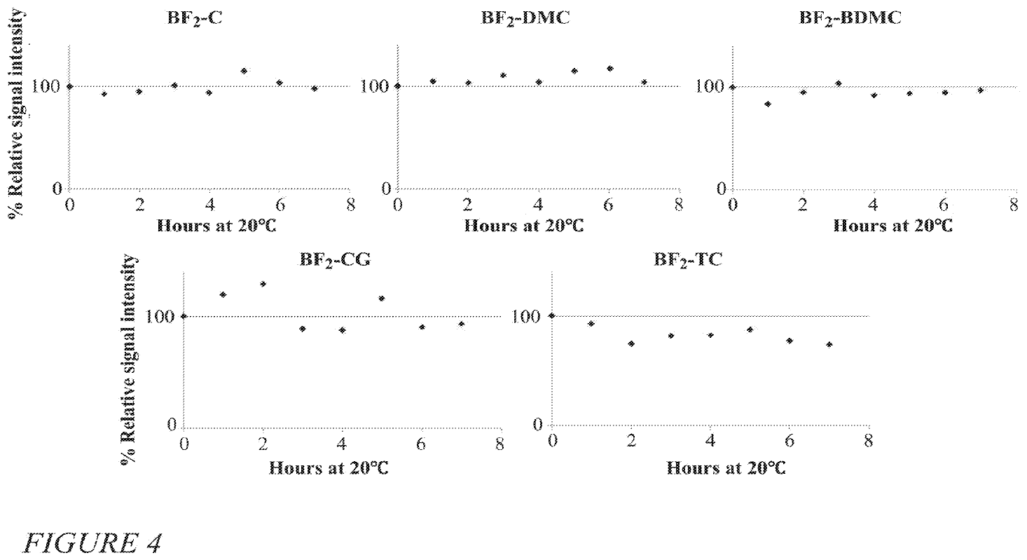

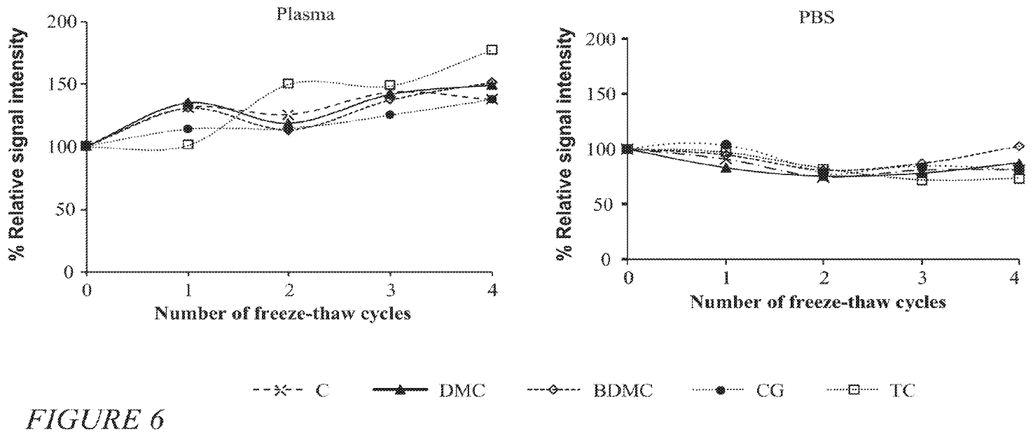

Freeze-thaw cycle stability in both PBS and plasma showed all curcuminoids to be stable after multiple freeze-thaw cycles with no sign of degradation (FIG. 6). The overnight stability of the BF₂-curcuminoid complexes in the LC autosampler showed stability in the autosampler throughout the tested duration (FIG. 4).

The following components and steps of the assay are described in further detail, but a skilled artisan will recognize that variations of the methods are within the scope of the invention. Various concentrations, amounts, ratios, durations, and other steps are merely guidance for achieving the results of the methods and variations that achieve the same outcome are fully embodied herein.

Preparation of standards. Stock solutions of the curcuminoids to be quantified in the assay are utilized. In one embodiment, C, DMC, BDMC, and TC are prepared in methanol (for example at concentrations of 100 nmol/mL). A CG stock solution at the same concentration is prepared in methanol/deionized H₂O (for example, 70/30, volume/volume). Working curcuminoid mixture solutions are prepared from the stock solutions by serial dilution in methanol. In addition, internal standards (for example, ²H₆-curcumin (d6C) and ²H₆-tetrahydrocurcumin (d6TC)) are prepared in methanol at a concentration of 0.1 and 0.5 nmol/mL, respectively. All these solutions may be stored at −20° C. in the dark.

Quality control samples. Quality control (QC) samples at three different concentrations (QC-low, QC-middle, QC-high) are used to evaluate and validate the properties of the assay. In one embodiment, QC samples are prepared by spiking appropriate volumes of the working curcuminoid standard mixture to 100 uL of curcumin-free human plasma. Absolute concentrations of C, DMC, BDMC in QC-low, QC-medium, QC-high samples are 0.5, 2.5, and 25 nM, respectively, while for TC and CG, 5, 25, and 100 nM, respectively.

Samples. Levels of curcuminoids are of interest in numerous biological samples, such as but not limited to bodily fluids, healthy and diseased tissues, plant materials, foodstuffs, cosmetics, nutritional and dietary supplements, flavorings, and the like. Such samples may be complex in having multiple components as often are present in biological specimens. In one embodiment, the bodily fluid or fraction thereof may be plasma, serum, whole blood, red blood cells, urine, lymphatic fluid, cerebrospinal fluid, saliva, sweat or semen. Tissue samples may be from any part of the body, and may be fresh, frozen, paraffin fixed, or preserved by other means. Tissue samples may be from a healthy or diseased organism, and may be, for example, tumor tissue. Dry or solid samples may be minced, pulverized, ground, shaken, or otherwise treated such that curcuminoids in the sample can be extracted into a liquid phase. Each type of sample may have a slightly different sample preparation method to be compatible with the subsequent steps of the assay method; these preparation methods will be readily carried out by one of skill in the art. Some examples of extraction methods are provided here but are not intended to be limiting. The following methods may be applied to plasma, red blood cells and soft tissue extracts, and may be easily modified for any other types of samples.

Extraction of human plasma samples. Plasma samples may be prepared in duplicate by transferring aliquots of human plasma (100 uL) to 1.5 mL microcentrifuge tubes. The internal standards (1 pmol d6C and 5 pmol d6TC in 10 uL of methanol) are added to each sample. They are briefly vortexed, then treated with cold 10 mM acetic acid in methanol (1 mL). After rigorous mixing, incubation (4° C., 60 min) and centrifugation (16,000 g, Rt, 5 min) supernatants are transferred to clean microcentrifuge tubes and are dried in a vacuum centrifuge. The dried samples are tightly capped and stored in −80° C.

Preparation of RBC and soft tissue samples. Aliquots (100 uL) of RBC or pre-weighed soft tissue samples are transferred to 1.5 mL microcentrifuge tubes. The internal standards (1 pmol d6C and 5 pmol d6TC in 10 uL of methanol) are added to each sample. 120 uL of 10 mM acetic acid in H₂O is added to each sample followed by addition of 500 uL of ethyl acetate/methanol (10/1) containing 0.1% butylated hydroxytoluene. Samples are homogenized by sonication, bead-beating, or other appropriate techniques. After homogenization, samples are centrifuged (16,000 g, Rt, 5 min), and top organic layers are transferred to clean microcentrifuge tubes and dried in a vacuum centrifuge. The dried samples are tightly capped and stored in −80° C.

Derivatization. After sample preparation, in one example, twenty uL of anhydrous acetic acid is added to the dried samples, followed by 15 seconds of sonication in a water bath sonicator. Then 20 uL of BF₃ reagent (20% boron trifluoride diethyl etherate (Acros Organics, Germany, code number 174560250) in anhydrous acetic acid, prepared fresh immediately prior to use) is added to each sample, which are then incubated (30 min, Rt in the dark), following which the samples are centrifuged (16,000 g, 5 min, Rt), and the supernatants carefully transferred to polypropylene HPLC injector vials for the LC-MS analysis. Alternately, boron trifluoride acetic acid or other forms or complexes of boron trifluoride may be used such as but not limited to boron trifluoride acetonitrile, boron trifluoride methanol, boron trifluoride ethylamine and boron trifluoride ethyl acetate.

Calibration curve standards. Simultaneously with each batch of biological samples, calibration curve standards are prepared in triplet at varying concentrations (0 and for example, 0.5, 1, 5, and 25 nM for C, DMC, and BDMC; 0, 5, 25, 50, 100 nM for CG and TC) by spiking the working curcuminoid mixture solution into curcumin-free human plasma (100 uL). After addition of the internal standards (1 pmol D6C and 5 pmol D6TC in 10 uL of methanol, same as above), the standards are processed concurrently with the biological samples.

LC-MS analysis. Aliquots (typically 15 uL) of the samples are injected onto a C18 reversed phase HPLC column (such as a Agilent Poroshell 120 SB-C18, 2.7 um, 150×2.1 mm, or equivalent) equilibrated in eluant A (water/formic acid, 100/0.01, v/v) and eluted at 100 uL/min with an increasing concentration of eluant B (acetonitrile/isopropanol (50/50, v/v: min/% B, 0/15, 1.5/15, 6.5/100, 7.5/100, 8/15, and 10/15). The effluent from the column is passed directly to an electrospray ion source connected to a mass spectrometer (such as Thermo LTQ XL) operating in the negative ion MSn mode, in which parent ions at m/z 415.1 (BF₂-C), 355.1 (BF₂-BDMC), 591.1 (BF₂-CG), 385.1 (BF₂-DMC), 419.1 (BF₂-TC), and 421.1 (BF₂-D6C) are selected and fragmented with previously optimized settings (isolation width 2.0, normalized collision energy 35, and activation Q 0.25). While BF₂-C, -BDMC, -CG, -DMC, and -D6C are monitored in MS2 scans, BF₂-TC is monitored in MS3 scan, in which the major MS2 fragment ion at m/z 283 is further fragmented again for improved specificity. Data is extracted from each file for the corresponding MS2 or MS3 fragment ions at m/z 400 (BF₂-C), 289 (BF₂-DDC), 415 (BF₂-CG), 147 (BF₂-TC), 370 (BF₂-DMC) and 403 (BF₂-D6C) using a 1 Da window. Standard curves for each compound are plotted as peak area ratio (analyte peak area/d6C peak area; ordinate) against concentration of each analyte in the sample (0, 1, 5, 10, or 50 pmol/mL; abscissa). The concentration of each analyte in the sample is thereby computed by interpolation using the standard curve of the corresponding analyte.

As noted above, the curcuminoids C, DMC, and BDMC have a calculated LoD of 0.01 nM and a calculated LoQ of 0.05 nM. In some embodiments, an LoQ of 0.06 nM, 0.07 nM, 0.08 nM, 0.09 nM, 0.1 nM, 0.2 nM, 0.3 nM, 0.4 nM or 0.5 nM is used. In some embodiments for analysis of biological samples, 0.5 nM is used as a practical LoQ.

The metabolites of curcuminoids CG and TC have a calculated LoD of 0.1 nM and a calculated LoQ of 0.5 nM. In some embodiments, an LoQ of 0.2 nM, 0.3 nM, 0.3 nM, 0.4 nM, 0.5 nM, 0.6 nM, 0.7 nM, 0.8 nM, 0.9 nM, 1 nM, 2 nM, 3 nM, 4 nM or 5 nM is used. In some embodiments, for the analysis of biological samples, 5 nM is used as a practical LoQ.

Thus, in some embodiments, the method is capable of detecting curcuminoids and their metabolites at a level of about 0.5 nM. In some embodiments, the method is capable of detecting curcuminoids and their metabolites at a level of about 0.1 nM. In some embodiments, the method is capable of detecting curcuminoids and their metabolites at a level of about 0.05 nM. In some embodiments, the method is capable of detecting curcuminoids and their metabolites at a level of about 0.01 nM.

Thus, in some embodiments, the method is capable of quantitating curcuminoids and their metabolites at a level of about 5 nM. In some embodiments, the method is capable of quantitating curcuminoids and their metabolites at a level of about 1 nM. In some embodiments, the method is capable of quantitating curcuminoids and their metabolites at a level of about 0.5 nM. In some embodiments, the method is capable of quantitating curcuminoids and their metabolites at a level of about 0.1 nM. In some embodiments, the method is capable of quantitating curcuminoids and their metabolites at a level of about 0.05 nM.

In some embodiments, the metabolites of glucuronidated DMC and BDMC (DMCG and BDMCG) are anticipated to have the same LoD and LoQ as CG.

In some embodiments, a method is provided for quantifying one or more curcuminoids and their metabolites present in a sample, the method comprising the steps:
(1) adding at least one internal standard to the sample or to an extract thereof;
(2) treating the sample or extract thereof with an organic solvent, mixing, optionally homogenizing, centrifuging, and collecting the organic solvent phase;
(3) drying down the organic solvent phase;
(4) derivatizing the one or more curcuminoids therein with boron trifluoride; and
(5) subjecting the sample to liquid chromatography separation and tandem mass spectrometry;
wherein the signal detected from each of the one or more curcuminoid in the sample or extract thereof compared to a signal from a standard, relative to the signal of at least one internal standard, indicates the quantity of each curcuminoid in the sample or extract thereof.

In some embodiments, a method is provided for quantifying one or more curcuminoids and their metabolites present in a sample, the method comprising the steps:
(1) adding one or more internal standards to the sample or extract thereof;
(2) treating the sample or extract thereof with acetic acid;
(3) treating the sample or extract thereof with an organic solvent, such as methanol or ethyl acetate/methanol;
(4) homogenizing the sample or extract thereof and mixing;
(5) centrifuging the sample or extract thereof and collecting the organic layer;
(6) derivatizing the one or more curcuminoids therein with boron trifluoride;
(7) subjecting the sample to liquid chromatography separation and tandem mass spectroscopy;
   wherein the signal detected from each of the curcuminoids in the sample or extract thereof compared to a signal from a standard, relative to a signal of at least one internal standard, indicates the quantity of each of the curcuminoids in the sample or extract thereof.

In some embodiments, a method is provided for quantifying one or more curcuminoids and their metabolites present in plasma, the method comprising the steps:
(1) adding at least one internal standard to the sample;
(2) treating the sample with methanol containing 10 mM acetic acid and collecting the supernatant;
(3) drying down the supernatant;
(4) adding glacial acetic acid and derivatizing the one or more curcuminoids therein with boron trifluoride; and
(5) subjecting the sample to liquid chromatography separation and tandem mass spectroscopy;
   wherein the signal detected from each of the curcuminoids in the sample compared to a signal from a standard, relative to the signal of at least one internal standard, indicates the quantity of each of the curcuminoid in the sample.

In some embodiments, a method is provided for quantifying one or more curcuminoids and their metabolites present in a red blood cell or soft tissue sample, the method comprising the steps:
(1) adding at least one internal standard to the sample;
(2) treating the sample with acetic acid;
(3) treating the sample with ethyl acetate/methanol (10/1, v/v) containing 0.1% butylated hydroxytoluene;
(4) homogenizing the sample and mixing;
(5) centrifuging the sample and collecting the top, organic layer;
(8) drying down the supernatant;
(9) adding glacial acetic acid and derivatizing the one or more curcuminoids therein with boron trifluoride;
(10) centrifuging the sample and collecting the supernatant; and
(11) subjecting the sample to liquid chromatography separation and tandem mass spectroscopy;
   wherein the signal detected from each of the curcuminoids in the sample compared to a signal from a standard, relative to a signal of at least one internal standard, indicates the quantity of each of the curcuminoids in the sample.

The following examples are presented in order to more fully illustrate the preferred embodiments of the invention. They should in no way be construed, however, as limiting the broad scope of the invention.

### EXAMPLES

### Example 1. Materials and Methods

Preparation of standard solutions: Stock solutions of curcumin (C, Sigma, St. Louis Mo.), Demethoxycurcumin (DMC, ChromaDex, Irvine Calif.), bisdemethoxycurcumin (BDMC, ChromaDex), and tetrahydrocurcumin (TC, Toronto Research Chemicals, Toronto, Canada) were prepared in methanol (100 nmol/mL). A curcumin β-D-glucuronide (CG, Toronto Research Chemicals) stock solution at the same concentration was prepared in methanol/dH2O (70/30). Working curcuminoid mixture solutions were prepared from the stock solutions by serial dilution in methanol. In addition, internal standards (²H₆-curcumin (d6C, Toronto Research Chemicals) and ²H₆-tetrahydrocurcumin (d6TC, Toronto Research Chemicals)) were prepared in methanol at a concentration of 0.1 and 0.5 nmol/mL, respectively. All these solutions were stored at −80° C. in the dark.

Quality Controls: Quality control (QC) samples at three different concentrations (QC-low, QC-middle, QC-high) were prepared by spiking appropriate volumes of the working curcuminoid standard mixture to 100 uL of drug-naïve human plasma. The final concentrations of C, DMC, BDMC in QC-low, QC-medium, QC-high samples are 0.5, 2.5, and 25 nM, while for TC and CG, the final concentrations were 5, 25, and 100 nM.

Biological samples: Human plasma. From a healthy volunteer who orally consumed an over-the-counter curcuminoid formulation (Longvida®, Verdure Sciences, Noblesville, Ind.), a qualified phlebotomist collected blood into plasma collection tubes containing K₂EDTA (BD vacutainer). The tubes were centrifuged (1000 g, 15 min), and plasma was collected and stored at −80° C.

Murine soft tissues. Mouse liver, brain, and tumor samples were collected from animals that had been administered a proprietary turmeric formulation (PHRAD 129, Aveta Biomics, Bedford, Mass.). After dissection, the tissues were weighed, frozen and stored at −80° C.

Extraction of human plasma samples and BF₂ complex formation: Plasma samples were prepared in duplicate by transferring aliquots of human plasma (100 uL) to 1.5 mL microcentrifuge tubes. The internal standards (1 pmol d6C and 5 pmol d6TC in 10 uL of methanol) were added to each sample. They were briefly vortexed, then treated with cold 10 mM acetic acid in methanol (1 mL). After rigorous mixing, incubation (4° C., 60 min) and centrifugation (16,000 g, Rt, 5 min) supernatants were transferred to clean microcentrifuge tubes and dried in a vacuum centrifuge. The dried samples were tightly capped and stored in −80° C.

Twenty uL of anhydrous acetic acid was added to the dried samples, followed by 15 seconds of sonication in a water bath sonicator. Then 20 uL of BF₃ reagent (20% boron trifluoride diethyl etherate (Acros Organics, Germany, code number 174560250) in anhydrous acetic acid, prepared freshly immediately prior to use) was added to each sample, which were then incubated (30 min, Rt in the dark), following which the samples were centrifuged (16,000 g, 5 min, Rt), and the supernatants were carefully transferred to polypropylene HPLC injector vials for the LC-MS analysis.

Extraction of mouse liver, brain and tumor tissue samples: Acetic acid (120 uL, 10 mM), ethyl acetate/methanol (95/5, 500 uL), and a solution of ISs (1 pmol D6C and 5 pmol D6TC in 10 uL of methanol) were added to pre-weighed (30-50 mg) frozen tissue samples in 2 mL reinforced microcentrifuge tubes (Fisher, part #15-340-162) containing ceramic beads. The samples were homogenized in a bead mill (thrice for 10 seconds each time at RT). The homogenate was vigorously mixed (5 min, RT), centrifuged (16,000 g, 5 min at RT), and the supernatant was transferred to microcentrifuge tubes and dried in a vacuum centrifuge. The dried samples were tightly capped and stored at −80° C.

Calibration curve standards: Simultaneously with each batch of biological samples, calibration curve standards were prepared in triplet at increasing concentrations (0, 0.5, 1, 5, and 25 nM for C, DMC, and BDMC; 0, 5, 25, 50, 100 nM for CG and TC) by spiking the working curcuminoid mixture solution to curcumin-naive human plasma (100 uL). After addition of the internal standards (1 pmol D6C and 5 pmol D6TC in 10 uL of methanol, same as above), the standards were processed concurrently with the biological samples.

LC-MS analysis of BF₂-curcuminoid complexes: Aliquots (typically 15 uL) of the samples were injected onto a C18 reversed phase HPLC column (Phenomenex Kinetex®, 1.7 m, XB-C18, 100 Å, 100×2.1 mm, or equivalent) equilibrated in eluant A (water/formic acid, 100/0.01, v/v) and eluted at 100 uL/min with an increasing concentration of eluant B (acetonitrile/isopropanol (50/50, v/v: min/% B, 0/15, 1.5/15, 6.5/100, 7.5/100, 8/15, and 10/15). The effluent from the column was passed directly to an electrospray ion source connected to a mass spectrometer (such as a Thermo LTQ XL) operating in the negative ion MSn mode, in which parent ions at m/z 415.1 (BF₂-C), 355.1 (BF₂-BDMC), 591.1 (BF₂-CG), 385.1 (BF₂-DMC), 419.1 (BF₂-TC), and 421.1 (BF₂-D6C) were selected and fragmented with previously optimized settings (isolation width 2.0, normalized collision energy 35, and activation Q 0.25). While C, BDMC, CG, DMC, and D6C were monitored in MS2 scans, TC and D6TC were monitored in MS3 scan, in which the major MS2 fragment ions at m/z 283 and 286, respectively, were further fragmented again for improved specificity. Data was extracted from each file for the corresponding MS2 or MS3 fragment ions at m/z 400 (C), 289 (DDC), 415 (CG), 147 (TC), 370 (DMC) and 403 (D6C) using a 1 Da window. Standard curves for each compound were plotted as peak area ratio (analyte peak area/IS peak area; ordinate) against concentration of each analyte in the calibration standard curve standards (abscissa). D6C was used as the IS for all compounds except TC, for which D6TC was used. The concentration of each analyte in the sample was computed by interpolation using the corresponding standard curve of each analyte.

### Example 2. BF₂ Complex Formation

The completion of the BF₂ complex formation was determined by comparing the mass and ¹H-NMR spectra of curcumin and the product after the reaction with BF₃ reagent. The MS spectra show a mass increase of 38 Da corresponding to loss of a proton and gain of a BF₂ moiety, and no residual signal for starting material was observed by MS (FIG. 2) or by ¹H-NMR (FIG. 3) after the reaction. The ¹H-NMR spectra also show downfield shifting of most proton signals after derivatization, explained by the electron withdrawing nature of the Lewis acid. Furthermore, the downfield shift of the signal at 10.087 ppm (assigned as the phenolic protons) explains the improved sensitivity of BF₂-C complex in negative ESI-MS due to the increased acidity of the phenolic proton coupled with stabilization provided by the Lewis acid complex of the resulting anion.

Complex stability: The BF₂-curcumin complexes slowly hydrolyze in water and water/methanol mixtures with a half-life of a few hours at 4° C. For applicability to an LC/MS assay on biological samples containing amounts of curcuminoids as low as 50 fmol/sample, and using an aqueous chromatographic mobile phase, it was necessary to use conditions that minimized hydrolysis. This breakdown was slowed but not averted by storing the samples at even lower temperatures (−80° C.) prior to analysis. The breakdown of the derivatives was averted by performing the reaction in glacial acetic acid and directly injecting the reaction mixture onto the LC column. Even after the injection of hundreds of samples there has been no noticeable deterioration of chromatographic reliability. Furthermore, by using a short 10 minute chromatographic gradient the hydrolysis of the complexes during chromatography was minimized. The stability of BF₂-curcuminoid complex was tested to validate the assay for overnight analysis. Eight QC-mid samples were prepared, stored tightly capped to minimize contact with atmospheric moisture in the autosampler at 20° C., and each sample was analyzed every hour for up to seven hours. Except for BF₂-TC, the curcuminoid complexes were stable throughout the 7 hour test period with no significant decline in peak intensity (FIG. 4). In the case of BF₂-TC, there was a slight loss of signal intensity during the 7-hours, but this would be corrected by use of the heavy isotope labeled internal standard (D6TC).

BF₂-curcuminoid mass spectral characteristics: The negative ion ESI mass spectra of the BF₂ complexes of C, D6C, BDMC, DMC, TC, D6TC, and CG all showed intense ions corresponding to the [M-H]⁻ parents (FIG. 1, Table 1). Following collision-induced dissociation (CID), each BF₂-curcuminoid produced unique product ions suitable for use in quantitative analyses (Table 1). Mass losses caused by fragmentation were 15 Da (CH₃) for C and DMC, 18 Da (C²H₃) for D6C, 66 Da (BF₂OH) for BDMC, 176 Da (glucuronic acid residue, C₆H₈O₆) for CG, and sequential losses of 136 Da (2-methoxy-4-methylphenol, C₈H₈O₂) and 139 Da (²H₃-2-methoxy-4-methylphenol, C₈H₅²H₃O₂) for TC and D6TC, respectively.

TABLE I
Summary of the mass spectral characteristics of the BF₂-curcuminoid derivatives.

| | Elemental | Observed | Observed | | |
|---|---|---|---|---|---|
| | composition | [M − H]⁻ | MS² and MS³ | Mass lost | |
| Compound | of BF₂-derivative | ion (m/z) | transitions | (Da) | Assigned loss |
| Curcumin (C) | C₂₁H₁₉O₆BF₂ | 415 | 415 −> 400 | 15 | CH₃ |
| ²H₆-Curcumin (D6C) | C₂₁H₁₃D₆O₆BF₂ | 421 | 421 −> 403 | 18 | C²H₃ |
| Demethoxycurcumin | C₂₀H₁₇O₅BF₂ | 385 | 385 −> 370 | 15 | CH₃ |
| (DMC) | | | | | |
| Bisdemethoxycurcumin | C₁₉H₁₅O₄BF₂ | 355 | 355 −> 289 | 66 | BF₂OH |
| (BDMC) | | | | | |
| Curcumin β-D- | C₂₇H₂₇O₁₂BF₂ | 591 | 591 −> 415 | 176 | glucuronic acid |
| glucuronide (CG) | | | | | |
| Tetrahydrocurcumin | C₂₁H₂₃O₆BF₂ | 419 | 419 −> | 136, 136 | C₈H₈O₂ |
| (TC) | | | 283 −> 147 | | |
| ²H₆- | C₂₁H₁₇D₆O₆BF₂ | 425 | 425 −> | 139, 139 | C₈H₅²H₃O₂ |
| Tetrahydrocurcumin | | | 286 −> 147 | | |
| (D6TC) | | | | | |
| Demethoxycurcumin | C₂₆H₂₅O₁₁BF₂ | 561 | 561 −> 385 | 176 | glucuronic acid |
| glucuronide (DMCG) | | | | | |
| Bisdemethoxycurcumin | C₂₅H₂₃O₁₀BF₂ | 531 | 531 −> 355 | 176 | glucuronic acid |
| glucuronide (BDMCG) | | | | | |

Relative response of C and BF₂-C: Although the BF₂ complexes have already been used as a way to purify curcuminoids and related compounds, as we attempted to modify the phenolic groups on curcumin via the Mitsunobu reaction, an observation emerged that the BF₂-curcuminoid complex is more prone to deprotonation at the phenolic group than the native counterpart. Following this observation, it also became evident that the BF₂-C complex exhibits a better response in negative ion ESI-MS. To compare the relative responses for underivatized and derivatized C, aliquots (100 uL) of a plasma extract were spiked with varying amounts of C. Some of the aliquots were taken to dryness in a vacuum centrifuge, resuspended in 50 uL of methanol, and analyzed for underivatized C using previously optimized conditions in the negative ion mode (parent m/z 367→fragment m/z 217). Other aliquots of the spiked plasma extracts were taken to dryness in a vacuum concentrator, derivatized with BF₃—OEt₂ and analyzed for BF₂-C as described above. The results from these matched series of samples showed an average 28-fold increase in peak area for the complex over the underivatized compounds across the range of 7.5 fmol to 15 pmol injected (FIG. 5).

### Example 3. Assay Validation

Extraction recovery: From human plasma, using the procedure described above, the recovery of the curcuminoids was determined by comparing peak intensities from the QC samples (QC-low, mid, and high) to peak intensities from curcumin-naïve control human plasma spiked post-extraction with corresponding amounts of standards. Both QC and post-extraction spiked samples were analyzed in triplicate at each concentration level. The extraction recovery from plasma was greater than 80% for all compounds, including the glucuronide, at all tested concentrations (Table 2, upper panel).

Table 2 is a summary of curcuminoid recovery efficiencies from human plasma (upper panel) and mouse cerebellar tissue (lower panel). The final concentrations of C, DMC, and BDMC in QC-low, QC-medium, QC-high samples were 0.5, 2.5, and 25 nM, respectively, while for TC and CG, the final concentrations were 5, 25, and 100 nM, respectively.

TABLE 2
Curcuminoid recovery efficiencies.

| | QC-low | | QC-medium | | QC-high | |
|---|---|---|---|---|---|---|
| Recovery from | concentration | % | concentration | % | concentration | % |
| human plasma | (nM) | recovery | (nM) | recovery | (nM) | recovery |
| Curcumin | 0.5 | 101.8 | 2.5 | 87.4 | 25 | 88.0 |
| Demethoxycurcumin | 0.5 | 95.1 | 2.5 | 89.1 | 25 | 87.8 |
| Bisdemethoxycurcumin | 0.5 | 101.8 | 2.5 | 86.6 | 25 | 92.0 |
| Tetrahydrocurcumin | 5 | 117.9 | 25 | 104.2 | 100 | 101.3 |
| Curcumin β-D- | 5 | 98.2 | 25 | 82.0 | 100 | 85.3 |
| glucuronide | | | | | | |

| Recovery from mouse cerebellum | Spiked amount | % recovery |
|---|---|---|
| Curcumin | 2.5 pmol/30 mg wet tissue | 80.8 |
| Demethoxycurcumin | 2.5 pmol/30 mg wet tissue | 97.6 |
| Bisdemethoxycurcumin | 2.5 pmol/30 mg wet tissue | 87.4 |
| Tetrahydroxycurcumin | 2.5 pmol/30 mg wet tissue | 97.6 |
| Curcumin β-D-glucuronide | 2.5 pmol/30 mg wet tissue | 10.8 |

The extraction recovery of the curcuminoids from brain tissue was determined by comparing peak intensities from pre- and post-extraction spiked curcumin-naïve mouse cerebellum tissue. The results show excellent recoveries (>80%) for all compounds except for the glucuronide conjugate (Table 2, lower panel). The poor recovery of CG is presumably attributable to the more hydrophilic nature of the compound resulting in poor partitioning into the ethyl acetate phase. Homogenizing tissue in different solvents in some cases improved the CG recovery, but resulted in loss of recovery for other compounds. For example, curcumin recovery was about 30% when the brain tissue was homogenized in methanol. Because of greater importance assigned to quantitation of the unconjugated curcuminoids, we decided to continue with using ethyl acetate to extract soft tissues.

Assay validation: accuracy and precision. These parameters were determined by analyzing nine independent QC samples (three each QC-low, QC-mid, and QC-high) along with calibration curve standards in the same batch using D6C as the sole internal standard. The measured concentrations of individual analytes in the QC samples were compared to the nominal concentration in corresponding replicates. Accuracy for all compounds at all concentrations tested was close to 100% except for TC, again indicating the need for dedicated internal standards for precise quantitation (Table 3).

TABLE 3
Summary of accuracy and precision validation analysis in human plasma.

| | Nominal | Calculated | | |
|---|---|---|---|---|
| | (nM) | (nM) | % accuracy | % CV |
| | QC-low | | | |
| Curcumin | 0.5 | 0.49 ± 0.03 | 98.41 | 10.54 |
| Demethoxycurcumin | 0.5 | 0.44 ± 0.03 | 88.9 | 13.22 |
| Bisdemethoxycurcumin | 0.5 | 0.50 ± 0.04 | 99.24 | 13.48 |
| Tetrahydrocurcumin | 5 | 6.84 ± 1.41 | 136.78 | 35.68 |
| Curcumin β-D-glucuronide | 5 | 5.20 ± 0.25 | 104.06 | 8.23 |
| | QC-medium | | | |
| Curcumin | 2.5 | 2.61 ± 0.06 | 109.19 | 3.88 |
| Demethoxycurcumin | 2.5 | 2.56 ± 0.19 | 102.21 | 8.4 |
| Bisdemethoxycurcumin | 2.5 | 2.36 ± 0.19 | 94.39 | 14.21 |
| Tetrahydrocurcumin | 25 | 32.06 ± 2.76 | 128.22 | 14.93 |
| Curcumin β-D-glucuronide | 25 | 23.61 ± 0.85 | 94.43 | 6.27 |
| | QC-high | | | |
| Curcumin | 25 | 26.42 ± 0.07 | 105.69 | 0.44 |
| Demethoxycurcumin | 25 | 25.15 ± 0.48 | 100.60 | 3.33 |
| Bisdemethoxycurcumin | 25 | 25.98 ± 1.39 | 103.91 | 9.25 |
| Tetrahydrocurcumin | 100 | 148.91 ± 10.98 | 148.91 | 12.78 |
| Curcumin β-D-glucuronide | 100 | 96.14 ± 6.36 | 96.14 | 11.46 |

Assay validation: carry over. This was determined by measuring peak areas of each analyte after solvent (glacial acetic acid) injections (15 uL) following multiple injections of QC-high samples and the most concentrated calibration curve standard. No detectable carry-over was observed. However, blank solvent injections are routinely included after every 10 sample injections to safeguard against the unlikely event of significant carry over.

Assay validation: limit of detection (LoD), limit of quantitation (LoQ), and practical LoQ. The general convention for estimating LoD or LoQ is by calculating the mean background signal from blank control samples plus 3 or 10 times the standard deviation of the mean, respectively. Using these formulae, the calculated LoD and LoQ of C, DMC, and BDMC were 0.01 nM and 0.05 nM, respectively. For TC and CG, the calculated LoD and LoQ were 0.5 nM and 1 nM, respectively. However, we used five to ten times the calculated LoQ as a practical LoQ (0.5 nM for C, DMC, and BDMC; and 5 nM for CG and TC) to ensure maximal reliability and accuracy of quantitation.

Assay validation: freeze-thaw cycle stability. Because samples (plasma and tissue) are frequently stored frozen at −80° C. until analyzed, it was important to determine the stability of the native compounds during multiple freeze thaw cycles. One pmol of each analyte was added to 100 uL of phosphate buffered saline (PBS) and human plasma, and the samples were subjected to multiple freeze (−80° C.) thaw (RT) cycles before being processed for LC-MS analysis. The experiment was conducted without the addition of IS and the results were based on absolute signal intensities. In both PBS and plasma, all curcuminoid and related compounds tested were stable after multiple freeze-thaw cycles, with no sign of degradation (FIG. 6).

### Example 4. Representative Chromatograms

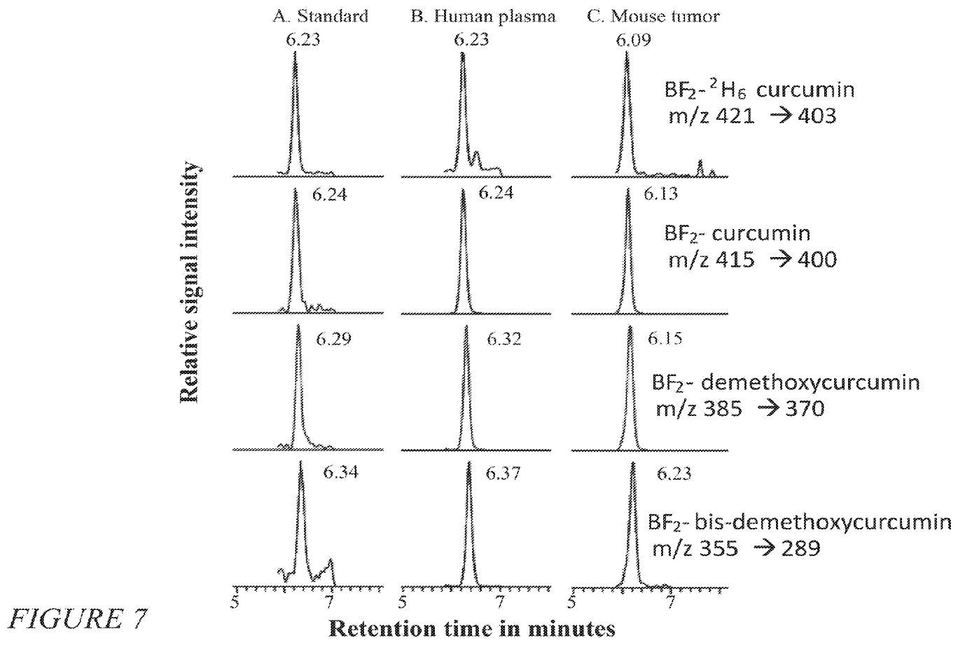

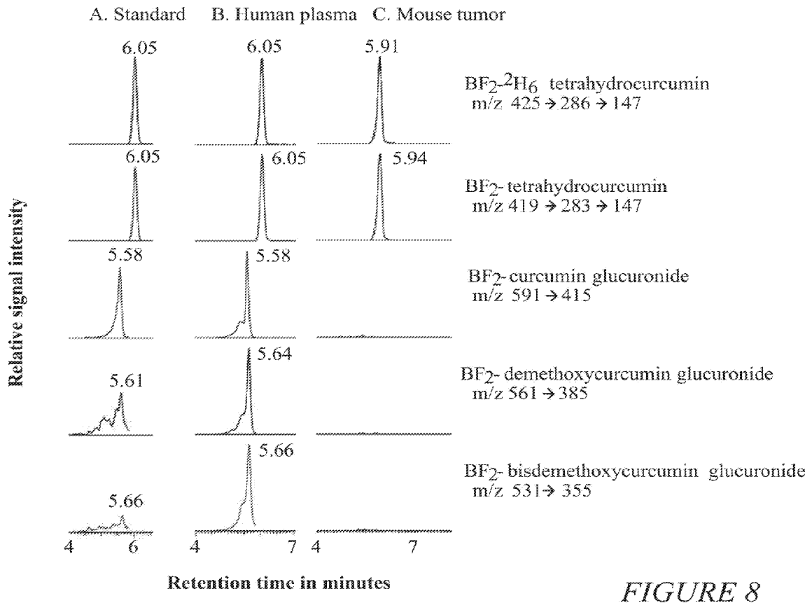

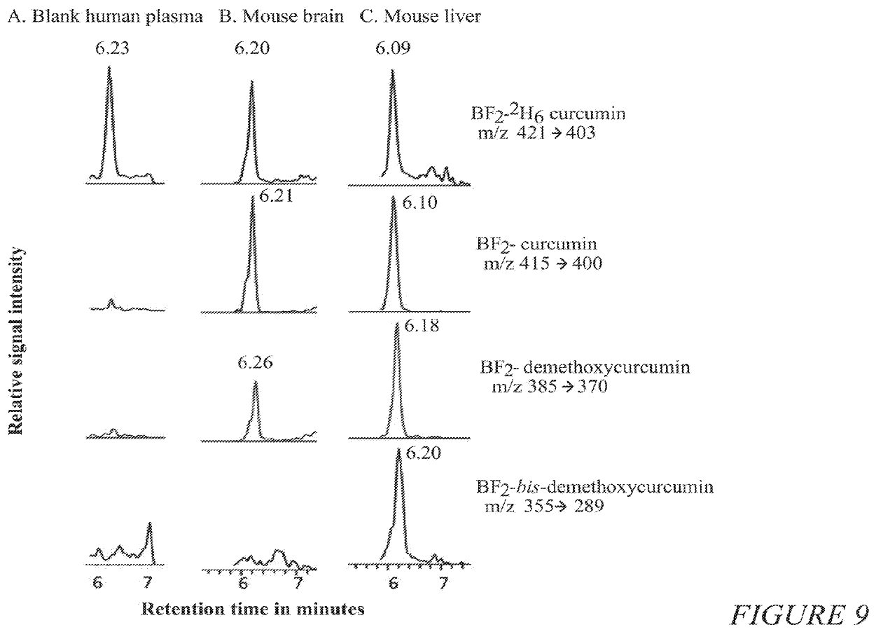

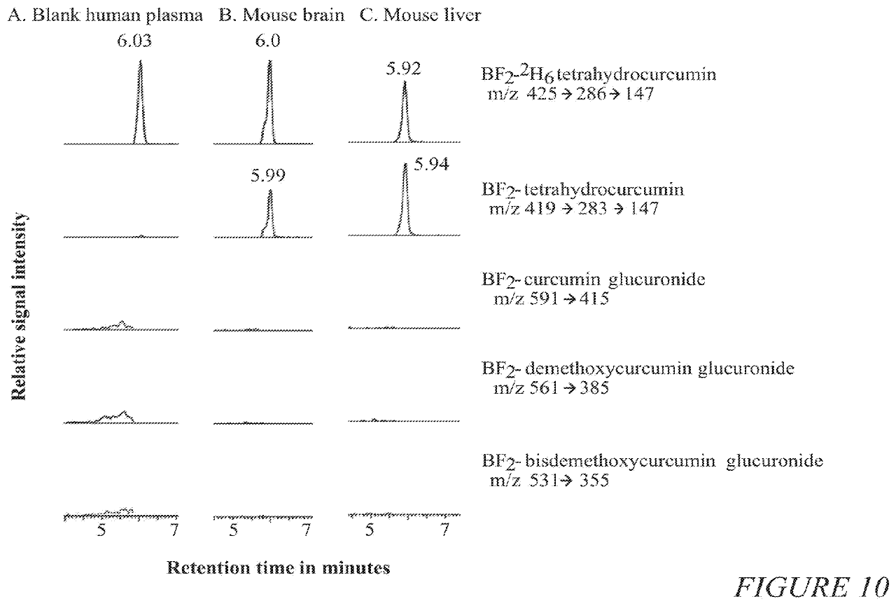

When injected at their practical LoQ in the milieu of human plasma, the curcuminoids (C, DMC and BDMC) and their metabolites (for which standards are available, TC and CG) all showed intense signals (FIGS. 7A and 8A, upper three traces, respectively). Extracts of plasma from a volunteer who orally consumed four tablets of a commercially available over-the-counter curcuminoid formulation (Longvida®, 230 mg of curcuminoids per 1000 mg tablet) daily for 17 consecutive days, showed strong signals for all three curcuminoids (FIG. 7B) and their metabolites (FIG. 8B, top three traces). An extract of a murine tumor following oral administration of a different turmeric formulation (PHRAD 129, dose=100 mg/kg, daily) showed strong signals for C, DMC, BDMC (FIG. 7C) and TC (FIG. 8C, upper two traces), although there was no peak for CG (FIG. 8C, third trace). Extracts from the brain (FIG. 9B) and liver (FIG. 9C) of mice that had been administered the same turmeric herbal supplement showed measurable peaks for C and DMC in brain and C, DMC, and BDMC in liver. There were no detectable peaks for any of the glucuronides in these samples (FIGS. 10B and 10C).

The loss of the glucuronic acid moiety (176 Da, Table 1, above) from CG during CID suggested that this could be a common feature of the fragmentation pattern of other curcuminoid glucuronides, and prompted the search for similar losses from the predicted molecular ions of the glucuronides of DMC, BDMC (Table 1) and TC. This search resulted in appearance in the same region of the chromatograms of intense peaks tentatively assigned as the glucuronides of DMC and BDMC (DMCG and BDMCG, respectively) in the resulting ion traces (FIG. 8B, lower two traces). However, peaks for these tentatively-assigned metabolites were only detected in the human plasma, and were not detected in the murine tumor (FIG. 8C lower two traces), brain or liver samples (FIGS. 10B and 10C, lower two traces).

### Example 5. In Vivo Temporal Profile of Plasma Curcuminoids and Metabolites in a Human Volunteer

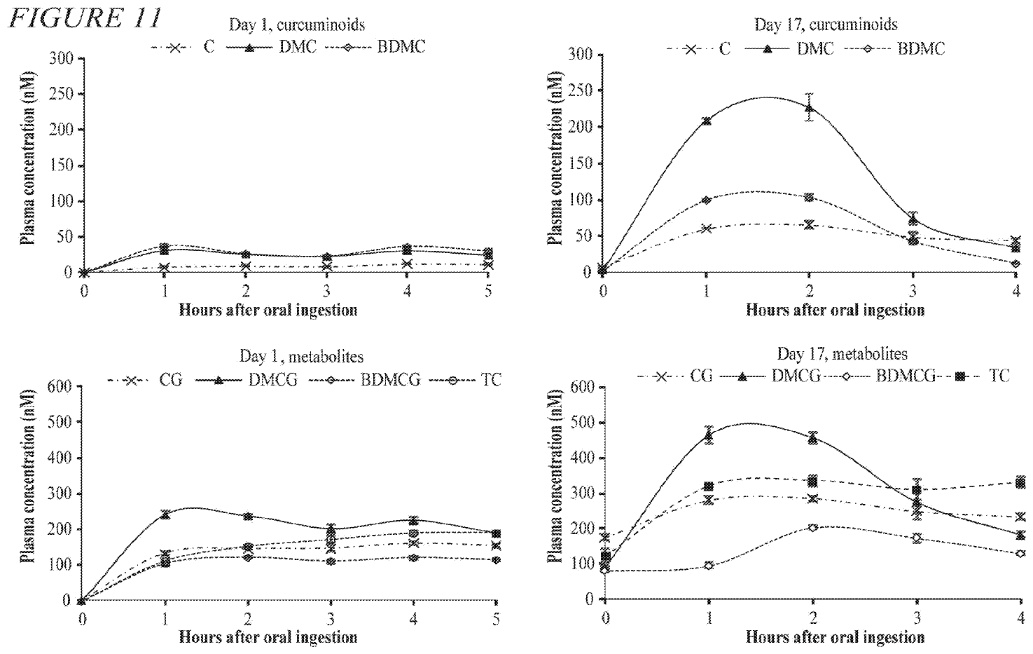

The in vivo pharmacokinetics of an orally administered curcuminoid formulation were explored in a single volunteer who orally consumed four tablets of a commercially available over-the-counter curcuminoid formulation (Longvida®, 230 mg of curcuminoids per 1000 mg tablet) each day for 17 consecutive days. Analysis of the blood collected at 0, 1, 2, 3, 4, and 5 hours after compound ingestion on Day 1 and 0, 1, 2, 3, and 4 hours on Day 17 was performed. The results show unexpectedly complex pharmacokinetic profiles (FIG. 11), presumably created in part by the time dependency of gastrointestinal absorption followed by appearance in plasma. Further complications might result from multiple elimination processes. It appears that all compounds (curcuminoids and their metabolites) were detected one-hour following drug administration on both Days 1 and 17. On Day 1, measured concentrations were up to 40 and 250 nM for the curcuminoids and metabolites, respectively; on Day 17 the concentrations were increased significantly up to 250 and 500 nM for the curcuminoids and metabolites, respectively. The metabolites were about 10-fold more concentrated than the curcuminoids on Day 1, but this difference fell to about 2-fold on Day 17. On Day 1 there is evidence of a biphasic temporal profile for both curcuminoids and the metabolites, but this reverts to a mono-phasic profile for all compounds on Day 17. There were significant circulating concentrations of the metabolites at the zero time-point on Day 17. Although these concentrations were low, they were still above the practical LoQ. The results from the Day 1 sample suggest the in vivo half-lives of all compounds (curcuminoids and metabolites) is significantly longer than 5 hours as there was no suggestion of a decline in their circulating concentrations during the first 5 hours following ingestion of the formulation. However, the time dependency of the appearance of the compounds in the plasma following entrance into the stomach and absorption into the plasma, already referred to, makes it impossible to calculate a realistic in vivo half-life from this experiment. Perhaps the most interesting aspect of the kinetics was the increase in bioavailability (area under the time versus concentration curves) by about 5-fold during the 17-day period. This aspect of the kinetics highlights the increase in bioavailability during the experiment, an increase that might be even greater were the regimen to continue beyond 17 days.

### Example 6. Validation of Method Using RBC

Extraction recovery: Recovery was determined by comparing the peak intensities from the QC samples (QC-low, -mid, and -high) to the curcumin-free human RBC spiked with corresponding amounts of each authentic compounds post-extraction. Both QC and post-extraction spike samples were analyzed in triplet at each concentration level. For C, DMC, BDMC, and TC, their extraction recovery ranged between 23-90% (Table 3). The extraction recovery of CG was exceptionally low at around 1-3%, which is expected as CG, which is conjugated with a sugar group, would not partition well into the organic solvent layer.

TABLE 4
Extraction recovery of curcuminoids and curcumin metabolites from red blood cells

| | Low | | Medium | | High | |
|---|---|---|---|---|---|---|
| | Concentration | % | Concentration | % | Concentration | % |
| | (nM) | recovery | (nM) | recovery | (nM) | recovery |
| Curcumin | 0.5 | 23.6 | 2.5 | 20.8 | 25 | 37.4 |
| Demethoxycurcumin | 0.5 | 79.3 | 2.5 | 34.5 | 25 | 79.6 |
| Bisdemethoxycurcumin | 0.5 | 52.2 | 2.5 | 90.6 | 25 | 90.1 |
| Tetrahydrocurcumin | 5 | 81.3 | 25 | 90.3 | 100 | 87.9 |
| Curcumin β-D-glucuronide | 5 | 3.3 | 25 | 2.0 | 100 | 1.1 |

Accuracy and precision of RBC assay. Accuracy and precision were determined by analyzing nine independent QC samples (triplets of QC-low, QC-mid, and QC-high) along with calibrator curve standards in the same batch. The measured concentration of individual analytes in the QC samples were compared to the nominal concentration in corresponding replicates to assess accuracy. The precision of the assay was calculated by comparing measured concentrations of each analyte among the same replicates. The data (Table 4) shows % Coefficient of Variation less than 15%, and an accuracy between 77-112% for all compounds except for CG. Poor extraction recovery is likely contributing to the inferior CG result.

TABLE 5
Red blood cell assay accuracy and precision

| | Nominal | Calculated | | |
|---|---|---|---|---|
| | (nM) | (nM) | % accuracy | % CV |
| | Low | | | |
| Curcumin | 0.5 | 0.51 ± 0.03 | 101.88 | 9.16 |
| Demethoxycurcumin | 0.5 | 0.52 ± 0.03 | 103.02 | 9.94 |
| Bisdemethoxycurcumin | 0.5 | 0.56 ± 0.05 | 111.25 | 14.96 |
| Tetrahydrocurcumin | 5 | 3.88 ± 0.17 | 77.63 | 7.54 |
| Curcumin β-D-glucuronide | 5 | 4.40 ± 2.62 | 87.91 | 103.04 |
| | Medium | | | |
| Curcumin | 2.5 | 2.61 ± 0.18 | 104.47 | 11.69 |
| Demethoxycurcumin | 2.5 | 2.79 ± 0.13 | 111.78 | 8.15 |
| Bisdemethoxycurcumin | 2.5 | 2.80 ± 0.14 | 112.11 | 8.36 |
| Tetrahydrocurcumin | 25 | 23.09 ± 0.64 | 92.38 | 4.78 |
| Curcumin β-D-glucuronide | 25 | 15.27 ± 4.51 | 61.07 | 51.22 |
| | High | | | |
| Curcumin | 25 | 23.72 ± 0.30 | 94.9 | 2.2 |
| Demethoxycurcumin | 25 | 22.78 ± 0.31 | 91.12 | 2.38 |
| Bisdemethoxycurcumin | 25 | 24.94 ± 0.32 | 99.76 | 2.23 |
| Tetrahydrocurcumin | 100 | 93.13 ± 4.78 | 93.13 | 8.89 |
| Curcumin β-D-glucuronide | 100 | 48.65 ± 8.76 | 48.65 | 31.18 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for quantifying one or more curcuminoids and their metabolites present in a sample, the method comprising the steps of derivatizing the curcuminoids in the sample to a boron difluoride curcuminoid complex, and quantifying the boron difluoride curcuminoid complexes by liquid chromatography-tandem mass spectroscopy, thereby quantifying the one of more curcuminoids present in the sample.

2. The method of claim 1 wherein the derivatizing is achieved by reacting the one or more curcuminoids in the sample with boron trifluoride.

3. The method of claim 2 wherein the boron trifluoride is in the form of boron trifluoride diethyl etherate.

4. The method of claim 1 wherein one or more curcuminoids or curcuminoid metabolites are curcumin, bis-demethoxycurcumin, demethoxycurcumin, curcumin β-D-glucuronide, tetrahydrocurcumin, demethoxycurcumin glucuronide, bisdemethoxycurcumin glucuronide, or other derivatives and metabolites of curcumin that retain the diketone moiety.

5. The method of claim 1 where at least one internal standard is added to the sample.

6. The method of claim 5 wherein the internal standard is ²H₆-curcumin or 2H₆-tetrahydrocurcumin.

7. The method of claim 1 wherein the sample is a bodily fluid or fraction thereof, a tumor sample or specimen, a tissue sample or specimen, a plant, a foodstuff or extract thereof, a cosmetic, a food supplement or flavoring, or a dietary supplement.

8. The method of claim 7 wherein the bodily fluid or fraction thereof is plasma, serum, whole blood, red blood cells, urine, lymphatic fluid, cerebrospinal fluid, saliva, sweat or semen.

9. The method of claim 1 wherein the steps comprise:
(1) adding at least one internal standard to the sample or to an extract thereof;
(2) treating the sample or extract thereof with an organic solvent, mixing, optionally homogenizing, centrifuging, and collecting the organic solvent phase;
(3) drying down the organic solvent phase;
(4) derivatizing the one or more curcuminoids therein with boron trifluoride; and
(5) subjecting the sample to liquid chromatography separation and tandem mass spectrometry;
wherein the signal detected from each of the one or more curcuminoid in the sample or extract thereof compared to a signal from a standard, relative to the signal of at least one internal standard, indicates the quantity of each curcuminoid in the sample or extract thereof.

10. The method of claim 1 wherein the steps comprise:
(1) adding one or more internal standards to the sample or extract thereof;
(2) treating the sample or extract thereof with acetic acid;
(3) treating the sample or extract thereof with an organic solvent, such as methanol or ethyl acetate/methanol;
(4) homogenizing the sample or extract thereof and mixing;
(5) centrifuging the sample or extract thereof and collecting the organic layer;
(6) derivatizing the one or more curcuminoids therein with boron trifluoride;
(7) subjecting the sample to liquid chromatography separation and tandem mass spectroscopy;
wherein the signal detected from each of the curcuminoids in the sample or extract thereof compared to a signal from a standard, relative to a signal of at least one internal standard, indicates the quantity of each of the curcuminoids in the sample or extract thereof.

11. The method of claim 8 wherein the steps for analysis of plasma comprise
(1) adding at least one internal standard to the sample;
(2) treating the sample with methanol containing 10 mM acetic acid and collecting the supernatant;
(3) drying down the supernatant;
(4) adding glacial acetic acid and derivatizing the one or more curcuminoids therein with boron trifluoride; and
(5) subjecting the sample to liquid chromatography separation and tandem mass spectroscopy;
wherein the signal detected from each of the curcuminoids in the sample compared to a signal from a standard, relative to the signal of at least one internal standard, indicates the quantity of each of the curcuminoid in the sample.

12. The method of claim 8 wherein the steps for analysis of red blood cells and soft tissues comprise
(1) adding at least one internal standard to the sample;
(2) treating the sample with acetic acid;
(3) treating the sample with ethyl acetate/methanol (10/1, v/v) containing 0.1% butylated hydroxytoluene;
(4) homogenizing the sample and mixing;
(5) centrifuging the sample and collecting the top, organic layer;
(8) drying down the supernatant;
(9) adding glacial acetic acid and derivatizing the one or more curcuminoids therein with boron trifluoride;
(10) centrifuging the sample and collecting the supernatant; and
(11) subjecting the sample to liquid chromatography separation and tandem mass spectroscopy;
wherein the signal detected from each of the curcuminoids in the sample compared to a signal from a standard, relative to a signal of at least one internal standard, indicates the quantity of each of the curcuminoids in the sample.

13. The method of claim 1 wherein the liquid chromatography comprises a gradient wherein solvent A is water/formic acid 100/0.01 (vol/vol) and solvent B is acetonitrile/isopropanol 50/50 (vol/vol), comprising:

| Time | % A | % B |
|---|---|---|
| 0 | 85 | 15 |
| 1.5 | 85 | 15 |
| 6.5 | 0 | 100 |
| 7.5 | 0 | 100 |
| 8 | 85 | 15 |
| 10 | 85 | 15 |

14. The method of claim 1 wherein the liquid chromatography utilizes a C18 reversed phase column.

15. The method of claim 1 wherein the curcuminoid is a metabolite of curcumin or of a related compound.

16. The method of claim 1 wherein the method is capable of detecting curcuminoids and their metabolites at a level of about 0.5 nM.

17. The method of claim 1 wherein the method is capable of detecting curcuminoids and their metabolites at a level of about 0.1 nM.

18. The method of claim 1 wherein the method is capable of detecting curcuminoids and their metabolites at a level of about 0.05 nM.

19. The method of claim 1 wherein the method is capable of detecting curcuminoids and their metabolites at a level of about 0.01 nM.

20. The method of claim 1 wherein the method is capable of quantitating curcuminoids and their metabolites at a level of about 5 nM.

21. The method of claim 1 wherein the method is capable of quantitating curcuminoids and their metabolites at a level of about 1 nM.

22. The method of claim 1 wherein the method is capable of quantitating curcuminoids and their metabolites at a level of about 0.5 nM.

23. The method of claim 1 wherein the method is capable of quantitating curcuminoids and their metabolites at a level of about 0.1 nM.

24. The method of claim 1 wherein the method is capable of quantitating curcuminoids and their metabolites at a level of about 0.05 nM.
